(19) United States
(12) Patent Application Publication
(10) Pub. No.: US 2024/0074695 A1
(43) Pub. Date: Mar. 7, 2024

(54) TREATMENT DEVICE AND METHOD FOR CREATING A TREATMENT PLAN MAP

(71) Applicant: Erbe Elektromedizin GmbH, Tuebingen (DE)

(72) Inventors: Yannick Daniel, Balingen (DE); Markus Enderle, Tuebingen (DE); Ovidiu Jurjut, Bensheim (DE); Elena Leonberger, Pfullingen (DE)

(21) Appl. No.: 18/457,596

(22) Filed: Aug. 29, 2023

(30) Foreign Application Priority Data
Sep. 7, 2022 (EP) 22194389.7

(51) Int. Cl. A61B 5/00; A61B 5/103; G16H 20/00

(52) U.S. Cl. CPC A61B 5/441; A61B 5/004; A61B 5/1032; A61B 5/742; G16H 20/00

(57) ABSTRACT

A treatment device allows a treatment of tissue with a treatment medium that does not directly show a tissue trace with manual as well as machine instrument guidance and under adjustment of a treatment plan map, due to preceding treatments. The treatment plan map represents the transformation rules for the treating person. The concept allows the adaption to different patients or patient collectives as well as the individualization of the creation of the treatment plan map with regard to the using treating persons.

### CROSS REFERENCE TO RELATED APPLICATION(S)

This application claims the benefit of European Patent Application No. 22194389.7, filed Sep. 7, 2022, which is incorporated herein by reference in its entirety.

### TECHNICAL FIELD

The invention refers to a treatment device for treatment of human or animal tissue, particularly for treatment of tissue surfaces, e.g. cervical intraepithelial neoplasia. Further the invention refers to a method for creation of a treatment plan map for treatment of human or animal tissue, particularly for treatment of cervical intraepithelial neoplasia.

### BACKGROUND OF THE INVENTION

A system for treatment of external skin areas of a patient is known from US 2019/0366117 A1. For this purpose a control unit is connected with a database in which tissue features and associated energy parameters are stored. Based on these stored data, the energy dosage for treatment of the skin is determined. In addition, the control unit can consider patient individual parameters for determination of the dosage, such as age or skin type. After output of the determined energy dosage, the success of the treatment is evaluated by means of checking specific skin features. If necessary the treatment is repeated with an adapted energy dosage. For this purpose the control unit can comprise a module for machine learning.

In addition, a system is known from EP 3 576 100 A1, which serves decision support in a medical therapy. For this purpose a generator is provided that provides, based on machine learning, a result prediction of a therapy adapted to a patient.

Automatic therapy planning in radiology is in addition described in EP 3 384 962 A1. Reference is made to US2012/283574 A1, EP 1 644 867 B1, US 2021/0042915 A1 and EP 2 237 190 A2 as further prior art.

For treatment of tissue, particularly tissue surfaces, plasma can be used that due to its low temperature and/or application duration does not or not visibly damage the tissue thermally. For the treating person it is thereby critical to recognize at which location the tissue has to be treated with which intensity, because therapy success is not visible with the naked eye (macroscopically), as is otherwise common. For example, cervical intraepithelial neoplasia can be treated in this manner.

Cervical intraepithelial neoplasia are typically identified in a staining test in which the tissue area to be examined is treated with acetic acid solution and subsequently with iodine potassium iodide solution, which results in a gradually fading acetic white or iodide negative discoloration of the tissue. The specific reaction to the coloration marks pathological tissue sections. If the treating person has recognized in this manner where he/she has to treat, he/she carries out the treatment accordingly more or less appropriate. For this reason the treatment result highly depends on the capabilities and the experience of the treating person. Excessively treated areas as well as insufficiently treated or irregularly/inhomogeneously treated areas can result.

Apart from the acetic acid iodine coloring technique, also other coloring techniques as well as additional methods can be used in order to mark or identify tissue having need for treatment and/or pathological tissue. For example, optical techniques such as optical emission spectroscopy (OES), optical coherence tomography (OCT), Raman spectroscopy or hyper-spectral or multi-spectral camera devices can be used. Similarly in some application cases it can be relied on that the tissue to be treated is recognized by the treating person or the medical camera system solely on the basis of visible distinctions. This depends on the kind of tissue to be treated as well as on the tissue modification to be treated. For simplifying the optical recognition by the treating person, illumination devices can be used having specifically adjusted spectral composition of the light for illumination of the tissue.

### SUMMARY OF THE INVENTION

It is one object of the invention to provide a treatment device as well as a method for creating a therapy recommendation in order to increase effectiveness of the treatment and patient safety.

This object is solved by means of a treatment devices and methods as described herein.

According to the invention, a treatment device is provided that comprises a diagnosis device and an application device. The diagnosis device comprises an image capturing device for capturing at least one image, an image sequence or a video of the tissue, e.g. cervical tissue. The image, the image sequence or the video is provided to the diagnosis device that is configured for identification of the position and the need for treatment of different sections of the tissue. The diagnosis device can be configured to recognize pathological conditions of the tissue and to create a treatment plan map with regard to different positions and needs for treatment of different zones of the tissue. The treatment plan map represents a location-dependent therapy recommendation.

The application device comprises an instrument for application of a treatment medium on the tissue. For example, the instrument can be a plasma instrument, particularly an instrument for creation of an argon plasma jet. The plasma is preferably not in thermal equilibrium. Preferably it comprises a gas temperature (i.e. temperature of the heavy parts; e.g. argon atoms in case of an argon plasma), which is remarkably lower than the electron temperature, so that human tissue can be touched at least in an interval of one or a few seconds by the plasma without being subject to a damaging tissue coagulation. The gas temperature is preferably adjusted, so that the tissue temperature can indeed increase, thereby however remains below the coagulation temperature of proteins (i.e. lower than 60° C.). Such plasma is denoted as cold plasma or warm plasma, contrary to a plasma with higher gas temperature resulting in thermal coagulation of the tissue.

The application device comprises in addition an apparatus for supply of the instrument. The apparatus is configured to provide the instrument with operating medium and energy. The operating medium can be the treatment medium itself or a medium from which the treatment medium is created under aid of energy. If the treatment medium is cold or warm argon plasma, the operating medium is argon, for example. Energy is then provided in form of high frequency voltage or high frequency current, for example.

Part of the application device is in addition a navigation device by means of which the location and the influence intensity of the treatment medium on the tissue can be controlled. For example, the influence intensity can be determined by the strength of the current, the amount of the voltage, the shape of voltage and current, the flow rate of the treatment medium, e.g. the plasma jet, and/or by the duration of the application of the treatment medium on a specific location of the tissue or additional parameters.

The navigation device can be configured to signal the need for treatment of a tissue section that is presently struck by the treatment medium to the treating person and thus provide a therapy recommendation. The need for treatment determines the desired influence intensity, whereby the two can be in a non-linear correlation. A need for treatment is also derived by a transformation rule from the location-dependent diagnosis, i.e. the diagnosis map. The transformation rule is a creation rule for the treatment plan map and represents a treatment model. The treatment model defines which treatment strength or treatment intensity belongs to which degree of pathology.

The navigation device can indicate the need for treatment optically, e.g. in form of a treatment plan map, acoustically, haptically or in any other manner. In addition, it can be provided that the navigation device influences the strength or intensity of the treatment medium, e.g. in order to weaken it during treatment of areas having low need for treatment and to strengthen it during the application of areas having high need for treatment. In case of an instrument manually guided by the treating person, he/she can therefore move the treatment medium over the tissue with a relatively uniform speed, whereby the navigation device controls the application intensity depending on the location. If the possibility of a modulation of the strength of the treatment medium is however limited, as it can be the case when a cold plasma is the treatment medium, the local adjustment of the treatment strength to the need for treatment has to and can be realized by the treating person by means of the guidance of the instrument.

Further embodiments of the navigation device are possible. For example, the navigation device can optically indicate the areas to be treated to the treating person and register as well as also indicate the carried out treatment of the areas to be treated. For this purpose the treatment plan map can be superimposed with an influence map in which the location and the strength of the carried out treatment are noted. Due to this superimposition of the treatment plan map with the influence map, a result map can be created that only comprises the areas of the tissue that are still to be treated. As an option, also over-treated areas can be contained in the result map.

In a further embodiment input means can be provided that allow the treating physician to manually modify the proposed treatment plan map in that he/she includes areas in the treatment plan map that have not been proposed for treatment by the navigation device. Similarly he/she can also eliminate areas from the treatment plan map. Likewise the physician can adjust the proposed dosages upwards or downwards in sections.

The image capturing device being part of the treatment device can be configured for capturing a sequence of images of a specific tissue section at different points in time. The different points in time can be frames captured in quick succession in the context of a video recording forming a video sequence. In addition, the image capturing device can also be used to record the respective tissue area at different examination points in time, e.g. with distance of days or weeks, and to make individual images or image sequences or video recordings.

In a simple embodiment the diagnosis device is configured to determine the pathological condition of the tissue based on specific reaction resulting from the coloring in a staining test of each point of the tissue captured in the image. For example, the coloring can be determined in a staining test in which the tissue is applied with Lugol solution, acetic acid solution or another colorant, such as an agent reacting on the presence of HPV-viruses. It is, however, also possible to use multiple images instead of one single image. For example, they can be captured at different examination points in time with a distance of days or weeks, from which the treatment progress can be determined. Also other techniques, e.g. optical techniques, such as optical emission spectroscopy (OES), optical coherence tomography (OCT), Raman spectroscopy or hyper-spectral or multi-spectral camera devices can also be used for this purpose.

It is also possible to capture multiple images in short time distance as image sequence, e.g. as video. The diagnosis device can be configured to determine therefrom the variation of the coloration in the images, e.g. the decay behavior of a coloration, and to assign a key figure to each point of the tissue captured in the images based on the time progress of the variation of the coloration, wherein the key figure characterizes the local pathology of the tissue. In this manner at first a data set is created that can be denoted as diagnosis map. The navigation device can be additionally configured to determine the required influence intensity from the individual points of the diagnosis map by means of the transformation rule and to create a treatment plan map in this manner.

The treatment plan map can be used for machine guidance of the instrument, for example. Then it serves to control a positioning system, e.g. a surgery robot, which guides the instrument. Alternatively, the instrument can also be guided by a treating person manually. For this purpose the treatment plan map can be shown on a display device, e.g. a display or a print, in order to signal the need for treatment of individual tissue sections to the treating person. It is in addition possible to superimpose the treatment plan map in form of Augmented Reality onto the view of the tissue in order to make the treatment locations and intensities visible. For example, for this purpose the treatment plan map can be superimposed onto a camera image of the cervical tissue to be treated, can be transferred in eyeglasses of a treating person or can be made visible in another manner, e.g. in that the treatment plan map is projected onto the tissue by means of a projector. The eyeglasses as well as the projector form in these cases the mentioned display device.

It is additionally advantageous, if the navigation device comprises a recording device that is configured to record the treatment dosage resulting from the influence of the treatment medium on the tissue. Such a recording of the treatment dosage can be achieved, for example, in that the location and the influence duration of the treatment medium on the tissue is captured by means of a camera, for example, and the applied treatment dosage is determined depending on the location, if necessary under consideration of the intensity of the treatment medium, i.e. the plasma jet. The navigation device can be configured to create an influence map in this manner. It is possible to visualize the achieved location-dependent treatment dosage, i.e. the influence map, by means of the display device. For this purpose the display device can comprise a display, VR-glasses or another device that indicates which dosage the treating person has already applied at which location.

Thereby it is particularly possible to configure the navigation device in a manner so that it superimposes the treatment plan map with the influence map and creates a result map in this manner. In the result map locations can be marked at which the achieved treatment dosage has reached the locally required dosage resulting from the treatment plan map. The marking can be done in that in the sufficiently treated areas the need for treatment visualized in the treatment plan map is effaced. In this manner the treating person can recognize that at this location no further treatment is required or can be potentially even detrimental. The result map is thus empty after sufficient treatment. In addition, the navigation device can be configured to mark locations at which an overtreatment threatens, i.e. the achieved dosage exceeds the required dosage, as danger zone for example in color, to reduce or block the flow of treatment medium or to create other warning signals, e.g. acoustically, haptically or optically perceptible signals. All in all, this means that the navigation device can be configured to show the achieved treatment dosage in an influence map. It can be in addition configured to display a superposition of the treatment plan map and the influence map in order to make still remaining areas that need to be treated optically visible for the treating person as result map.

The diagnosis device can be connected with a data storage that is configured to specifically store the diagnosis of a patient or multiple patients. Particularly, the diagnosis device can be configured to create a diagnosis map based on an individual image or an image sequence that has been captured during an examination, e.g. during a stain test. The diagnosis device and/or the navigation device can be configured to create a treatment plan map based on the diagnosis map. The diagnosis map and/or the treatment plan map can be provided in a storage in a manner specific to the patient and can be, e.g. ordered by examination dates. In addition, the transformation rule (i.e. the creation rule), based on which the treatment plan map is created from the diagnosis map, can be stored in the storage.

In addition the navigation device can be configured to store the diagnosis map and/or the treatment map in a storage and to assign it/them to the examination dates of the patient or to additional data. Additional data can be of nature specific to the patient and/or treating person. Such data are, for example, the age of the patient, the physical overall condition of the patient, additional diseases of the patient, risk factors of the patient, etc.

The navigation device can be in addition configured to create an influence map that represents the applied location-dependent dosage of the treatment medium. The navigation device can be configured to transfer the influence map with the recorded influence intensities to the storage medium. The storage medium is preferably configured to store the influence map in a patient-specific manner and assigned to the treatment dates.

In a particularly preferred embodiment the data storage is connected with a treatment monitor. The latter is configured to compare the diagnosis map of a subsequent examination with the diagnosis map of a previous examination in order to determine treatment success.

In addition, the treatment monitor can be configured to correct the correlation between the diagnosis map and the treatment plan map. For this purpose the transformation rule can be modified, for example. In the simplest case the creation rule is an assignment of individual points of the diagnosis map to points of the treatment plan map having the same coordinates. A healthy point in the diagnosis map is assigned to a point in the treatment plan map that has no need for treatment. A point in the diagnosis map having a tissue characterization with need for treatment, e.g. an intraepithelial neoplasia, is assigned to a point in the treatment plan map with a determined influence intensity. This is a I/O-assignment. In a more refined treatment model a linear assignment between the degree of the pathology and the influence intensity can be provided. Also a non-linear assignment can be provided, e.g. in that as from a specific degree of the pathology, the influence intensity increases at a lower rate, e.g. in order to avoid tissue damages. When determining the required influence intensities in the treatment plan map, it is in addition possible to not only consider the assigned point having the same coordinates in the diagnosis map, but also consider points in the vicinity.

The treatment monitor can be configured to improve the transformation successively from the comparison of the diagnosis maps at different examination dates and the consideration of the influence maps. A first treatment plan map results from the diagnosis map of a first examination. The applicant then carries out the treatment based on the treatment plan map, whereby its treatment activity is recorded in the influence map. The diagnosis map captured in the second (subsequent) examination shows the result of this influence. In the ideal case this map is empty, i.e. it does not show any pathological modifications, e.g. no intraepithelial neoplasia. If pathological modifications, such as intraepithelial neoplasia, are still present, again a treatment plan map is created on basis of which a further treatment is carried out. The diagnosis maps created in the second or also in further examinations are the basis for a machine learning process in the context of which the transformation rule can be improved in a patient-specific or general manner.

In addition, the transformation rule can be improved in a manner specific to the treating person, because it can be assumed that the influence map deviates from the treatment plan map in a treating-person-specific manner. The treating-person-specific individual deviations are typically not solely stochastic, but depend from the individual movement motor skills of the treating person. The machine learning algorithm forms a feedback control loop, the controlled system of which comprises the treating person. The non-stochastic deviation between the treatment plan map and the influence map introduced by him/her is eliminated by means of the machine learning algorithm in the context of a feedback control process.

Also modifications in the proposed treatment plan map introduced manually by the user via an input device can be detected and evaluated by the learning algorithm in this sense in order to use them during the creation of the transformation rule.

The navigation device can be configured to create patient-related and/or treating-person-specific treatment models (transformation rules) and to use them in a patient-specific and/or treating-person-specific manner. In this manner patient-specific factors as well as individual practices of the treating person during the manual guidance of the instrument can be eliminated that could otherwise result in at least partly non-appropriate influences on the tissue. Due to the treating-person-specific treatment models (transformation rules), treating-person-specific treatment plan maps are created that balance tendencies of the treating persons, e.g. that tend to prefer specific areas or that otherwise tend to excessive or insufficient treatment.

In another embodiment multiple devices, according to the invention, are connected with one another by means of a network, e.g. a cloud network, and can thus exchange optimized transformation rules. In addition, the collected treatment data of networked devices can serve as basis for neuronal networks or other machine learning methods in order to obtain further optimizations of the transformation rules. It is in addition possible to provide particularly successful treatment models in a central storage for access. For example, a largely optimized treatment model can be created by means of the learning algorithm or treating person who treats very many similar pathological modifications of tissue. This treatment model can then be transferred to treatment devices and can be used there, also if similar treatments have to be carried out there only rarely. In thus far the device according to the invention allow an experience-transfer of highly specialized treating persons to less specialized treating persons without the need that the latter have to go through a learning process themselves. The system learns for them.

### BRIEF DESCRIPTION OF THE DRAWINGS

In the drawings the invention is illustrated in different aspects. The drawings show:

FIG. 1 the treatment device according to the invention in a block diagram illustration,

FIG. 2 a diagnosis map with a two-dimensional illustration of (pathological) areas of the cervical tissue that need to be treated,

FIG. 3 a treatment plan map having a two-dimensional illustration of areas having need for treatment,

FIG. 4 an influence map during creation having an identification of already treated areas,

FIG. 5 a treatment result map during creation having a two-dimensional illustration of remaining areas having need for treatment,

FIG. 6 the logical progress of the treatment and the treatment optimization in form of a flow diagram and

FIG. 7 a logic plan of the operation of the treatment device.

### DETAILED DESCRIPTION OF THE DRAWINGS

The device according to aspects of the invention and the method according to aspects of the invention are explained in the following by way of example of a treatment of cervical intraepithelial neoplasia. It can however also be used for other organs, e.g. the intestine and/or other body regions.

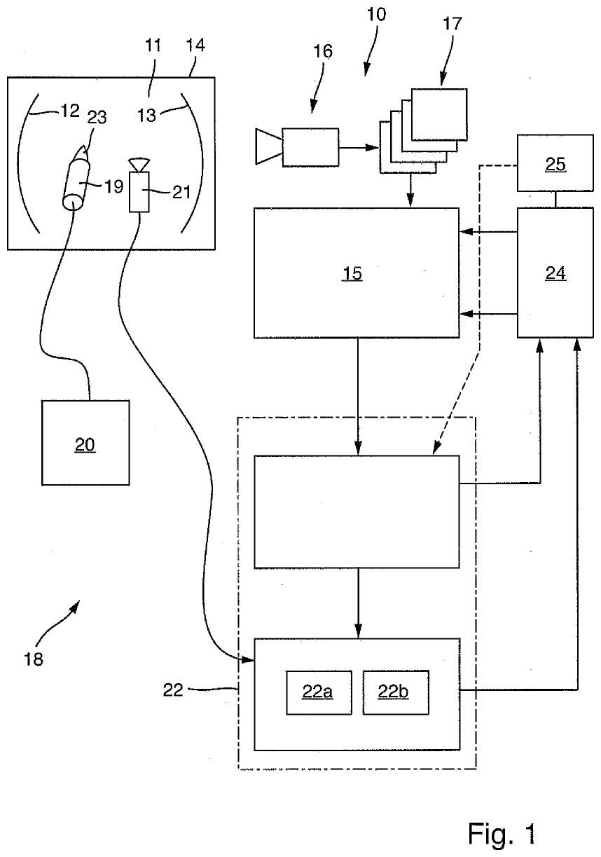

FIG. 1 illustrates a treatment device 10 according to the invention for treatment of cervix tissue 11, the access to which is maintained open by means of lamellae 12, 13 of a speculum. In an image 14 captured by means of a camera, the cervix tissue 11 is visible. With a respective image size also the lamellae 12, 13 can be visible in the image 14.

Part of the treatment device 10 is a diagnosis device 15 that is communicatively connected with an image capturing device 16, e.g. in form of a camera. The image capturing device 16 is configured to capture one or multiple images of the cervix tissue 11 and to transfer it/them in form of an image, a sequence of individual images or as video stream to the diagnosis device 15.

In addition, the treatment device 10 comprises an application device 18 having an instrument 19 for influencing cervix tissue 11, an apparatus 20 connected with the instrument 19 for supply of the instrument 19, an image capturing device 21, e.g. in form of a camera, and a navigation device 22.

The instrument 19 serves for application of treatment medium 23 on the tissue 11. The treatment medium 23 is preferably a treatment medium that, during treatment of the tissue, does not leave any directly visible traces thereon, such as electromagnetic radiation or a plasma, having an energy respectively that does not result in visible tissue coagulation and thus discoloration. Particularly a plasma with non-equilibrium properties can be used as plasma, such as a cold plasma or another plasma, the gas or ion temperature of which is so low that it does not result in a direct devitalization by means of coagulation of the tissue 11, also in case of influence during one or a few seconds. The ion temperature of the plasma is preferably below 70° C. or 60° C. and further preferably below 50° C., in the ideal case below 40° C.

The apparatus 20 supplies instrument 19 with the required operating media, e.g. argon and electromagnetic energy, such as HF-current for creation of a plasma having non-equilibrium properties that strikes on tissue 11 as treatment medium 23.

The instrument 19 is preferably hand guided and used by the treating person in order to guide the treatment medium 23 in a stripe-wise manner over the tissue 11. He/she can do this under control of the naked eye or under control of camera 21. Besides camera 21 can be identical with camera 16 or can be configured as separate camera. The instrument 19 can, however, also be guided in remote-controlled manner manually or automatically.

The treating person can observe his/her action in the context of which he/she applies tissue 11 with the treatment medium 23, as mentioned, with the naked eye, by means of camera 21 on a display device, e.g. a screen, or by means of other means, e.g. VR-glasses in which similar to an image on a screen additional information can be shown. Such additional information is provided by the navigation device 22. The additional information can particularly comprise a therapy proposal.

The treatment device 10 is preferably connected with a data storage 24 that is arranged in the proximity of the treatment location or also remote therefrom only in communication via a data transmitting network. The data storage 24 can be configured to store data and if necessary also process data provided by the diagnosis device 15 and the navigation device 22. A treatment monitor 25 can be connected to the data storage 24, wherein the treatment monitor 25 is configured to evaluate the success of a treatment and if necessary, provide data for support of the image analysis. In addition, the treatment monitor can influence the navigation device in order to influence the treatment to be carried out. The treatment monitor can be realized as hard- and/or software that monitors the treatment.

The subsequent description of the functions of the diagnosis device 15 and the navigation device 22 can run on a suitable programmable system, such as one or more computers, that are configured to carry out the indicated functions, e.g. by means of suitable programs. In so far, the following function description is also a description of the configuration of the treatment device 10.

On the basis of the above-described basic structure of the treatment device 10, it operates as follows:

Prior to the real treatment step cervix tissue 11 is first examined, the result of which forms the basis of the subsequent real treatment. For this a suitable examination liquid, such as Lugol solution or acetic acid or the like is applied on cervix tissue 11. Typically cervical intraepithelial neoplasia or their precursors are caused by HPV-viruses. Due to the HPV infestation, dysplastic tissue is made visible by means of the staining test. For example, pathologically modified tissue is thereby colored in white, wherein the white coloring decays over time.

For the treatment of other organs and/or body regions, other techniques can be used in order to make the tissue to be treated detectable for the diagnosis device 15.

The diagnosis device 15 can be configured to evaluate an individual image of the colored tissue 11 in order to determine the circumference (i.e. the location and the shape) of the infected area recognized due to the white coloring. Also the diagnosis device 15 can be configured to detect the location specific intensity of the white coloring. Alternatively or additionally, the diagnosis device 15 can be configured to evaluate multiple images 17 or a video sequence recorded during the staining task. The diagnosis device can be configured to conclude from the decay behavior, i.e. the fading of color of the stained area, the degree of the pathology, that is for example, the severity of the HPV infestation and/or the degree of the presence of intraepithelial neoplasia and thus the need for treatment of the tissue. The diagnosis device 15 can be configured to transmit the gained data in a time- and patient-specific manner to the data storage 24. From the data storage 24 the diagnosis device 15 can get information, for example about previous examinations or patient-type-dependent decay behavior, staining solution typical tissue reactions and the like. In FIG. 1 this is symbolized by means of arrows between data storage 24 and diagnosis device 15.

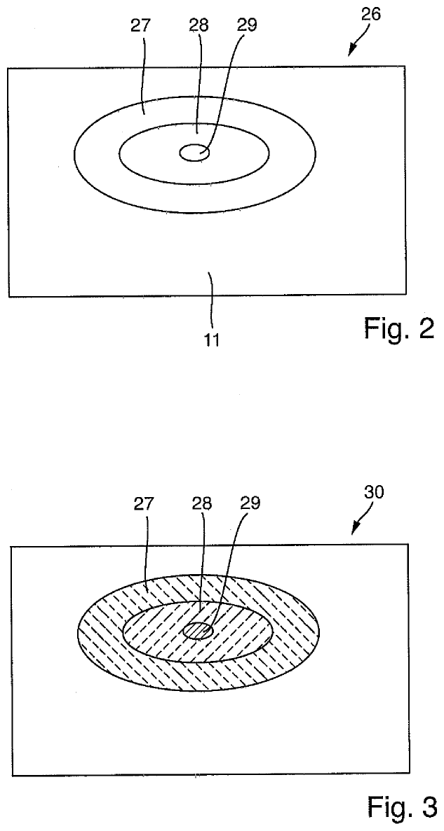

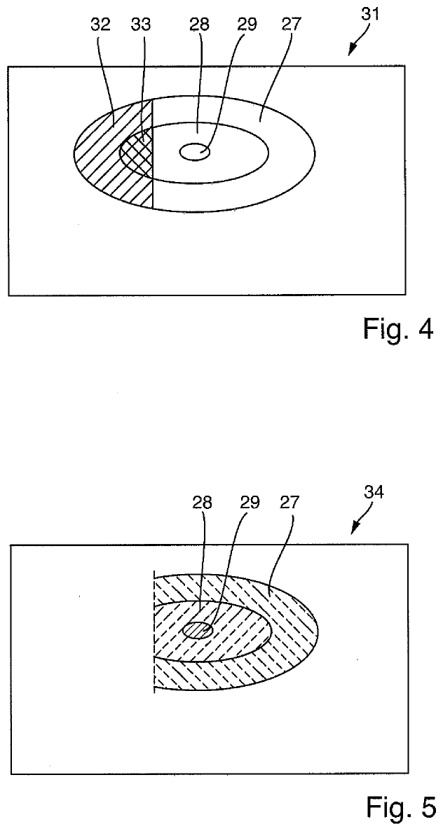

The diagnosis device is configured to first create a diagnosis map 26, as illustrated in FIG. 2. It can be considered as two-dimensional illustration of cervix tissue 11 in which at least one and if applicable also multiple zones 27, 28, 29 are marked that illustrate tissue infected by HP-viruses and/or already present cervical intraepithelial neoplasia. The zones 27, 28, 29 can illustrate different severity degrees of the infestation or neoplasia. The diagnosis map 26 can be directly displayed on a monitor or alternatively also processed internally only.

The diagnosis map 26 is the basis for the creation of the treatment plan map 30, as illustrated in FIG. 3. The treatment plan map 30 now shows the need for treatment for the zones 27, 28, 29. The treatment plan map 30 can be created solely by the navigation device 22 based on the diagnosis map 26 or also with additional access to patient data. For example, during creation of the treatment plan map 30 the history of the respective patient, e.g. in form of the number and intensity of the already undergone treatments, can be used. Also age and the other health status of the patient can be considered.

The creation of the treatment plan map 30 from at least the diagnosis map is carried out by means of a treatment model, which is a transformation rule. In the simplest case, the navigation device 22 assigns a need for treatment to the zones 27, 28, 29, which is proportional to the severity of the present lesion (HPV infestation or present neoplasia) for creation of the treatment plan map 30. The correlation defined by the transformation rule (i.e. the treatment model) can however also be carried out according to a non-linear function and, as mentioned above, under additional consideration of the treatment history.

In a preferred embodiment input means, e.g. in form of a keyboard, a computer mouse, a touchscreen, a voice control, etc., can be provided by means of which the treating person can manually modify the proposed treatment plan map 30. For this he/she can add tissue areas in the treatment plan map that have not been proposed for treatment by the diagnosis device. He/she can also eliminate areas from the treatment plan map. Similarly the treating person can adjust the proposed dosages zonally, e.g. upwards or downwards.

The treatment plan map 30 is represented to the treating person on a suitable representation device, e.g. by means of a screen or VR-glasses or is projected by means of a projector directly on the cervix tissue 11. This happens after examination during the actual treatment during which the treating person applies the treatment medium 23 on the tissue 11 by means of instrument 19. This acts on the tissue 11, which becomes however not directly visible on the tissue 11. The navigation device 22 is configured to detect the location, the movement and the influence duration of the treatment medium 23 over and on the tissue 11 and—at least in case of non-constant intensity of the treatment medium 23—also to consider the intensity thereof. Optionally navigation device 22 can be communicatively connected with apparatus 20 in order to use its operation data to determine and consider the intensity of the treatment medium 23 and also in order to set the intensity of the treatment medium 23. For this purpose navigation device 22 can comprise a recording device 22a.

From the registered trace of the influence of the treatment medium 23 on tissue 11, navigation device 22 creates an influence map 31 during treatment. The navigation device 22 is configured to superimpose the zone of influence 32 and if necessary, the zone of higher influence 33 onto zones 27, 28, 29. It is not necessary to indicate influence map 31 to the treating person. The navigation device can, however, be configured to create a result map 34 therefrom in which those portions of the zones 27, 28, 29 are eliminated that have already been subject to sufficient treatment with treatment medium 23. The treatment plan map 30, the influence map 31 and/or the result map 34 can be represented by means of a display device 22b, being part of the navigation device 22 individually or superimposed with a live image of camera 21. The display device can be, for example, VR-glasses, a monitor or similar.

The result map 34 can be displayed to the treating person by means of one of the above-mentioned suitable display media (projector, monitor, VR-glasses, etc.). The treating person will, therefore, guide the instrument 19 over zones 27, 28, 29 in a stripe-wise manner until they disappear from its field of view. At the beginning of the treatment the map indicated to the treating person is identical with the treatment plan map 30 according to FIG. 3 and represents a therapy recommendation. During treatment the treated areas are effaced, as illustrated in FIG. 5. At the end of the treatment the map is empty, all zones 27, 28, 29 have been effaced to indicate the completed treatment. The result map 34 can be formed as difference from treatment plan map 30 and influence map 31. If the treatment plan map 30 and the influence map 31 are identical, i.e. a treatment has been carried out exactly according to the plan, result map 34 is empty (i.e. zero everywhere). If deviations exist between influence map 31 and treatment plan map 30, they are documented in the result map.

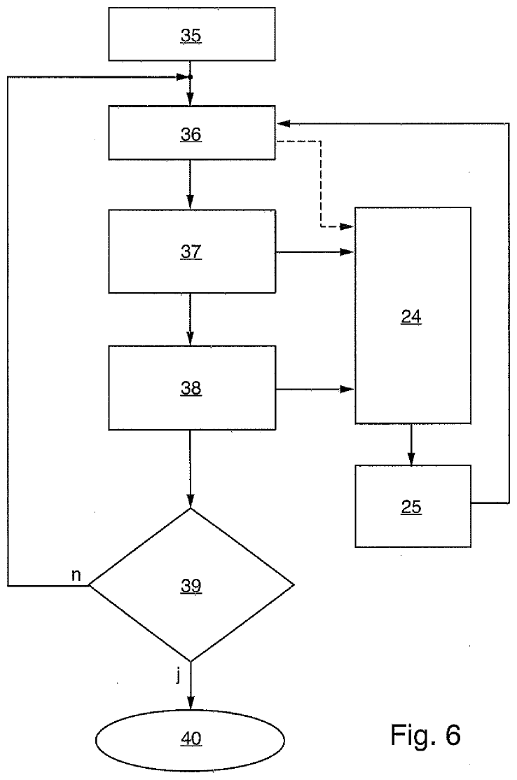

FIG. 6 illustrates the operation of the treatment device 10 in logic blocks as flow diagram. The progress starts in block 35 with the examination of the cervix tissue 11 of the patient, as described above. In block 36 diagnosis device 15 at first creates diagnoses map 26 from the at least one captured image 17 or an image sequence and therefrom under aid of the transformation rule (i.e. the treatment model) treatment plan map 30 according to FIG. 3. The transformation rule is stored in the navigation device 22 can consist, for example, in adjusting the influence intensity locally to the severity of the lesion, e.g. proportionally. Alternatively to this linear assignment between severity of the lesion and influence intensity, also a non-linear correlation can be selected, e.g. in order to avoid tissue damage due to too high influence intensity in case of severe lesions.

Based on the treatment plan map 30, the treating person can now carry out the treatment according to the treatment plan map 30 in block 37. Therewith a present treatment of the patient ends in the first instance. The treatment plan map 30 representing the therapy proposal and/or the influence map 31 documenting the actually carried out treatment can both be stored in the storage 24. In addition, the treatment model can be stored in storage 24.

To a later point in time the patient can again show up for examination, as symbolized by block 38. Thereby the carried out staining test is documented and evaluated again and a diagnosis map 26′ is created by means of diagnosis device 15.

The influence map 31 (and/or result map 34) transferred by block 37 to data storage 24 and the diagnosis map 26′ created during the new examination in block 38 by means of the diagnosis device 15 can be stored in storage 24. By means of comparison of the first diagnosis map 26, the treatment plan map 30, the influence map 31 and the diagnosis map 26′ gained in step 38, the treatment monitor 25 can determine whether a transformation rule leading from the diagnosis map 26 to the treatment plan map 30, i.e. the treatment model, can or has to be improved:

For example, it can become apparent that the zone 27 and the zone 29 have been sufficiently treated while areas having need for treatment still exist in zone 28. The treatment monitor 25 can be configured to determine this and to provide a correction recommendation for the transformation rule from diagnosis map 26′ to the treatment plan map. In the mentioned example the transformation rule (treatment model) for lesions of the degree according to zone 28 is modified in a way that an increased influence intensity is determined. The modification of the transformation rule gained in this manner can be determined for the specific patient or also for a larger patient collective, e.g. patients with equal or similar age, equal history (number of children, general physical status, weight, constitution, etc.).

If in block 38 and 39 a complete treatment success has been determined, the method ends in block 40. If not, the method is continued in block 36.

The progress according to FIG. 6 considers the function of treatment device 10 and thus the treating person. Because the treatment according to the treatment plan map 30 is carried out by a treating person, i.e. a human being, in block 37 inaccuracies have to be considered here. For example, treating persons can tend to treat zones 27, 28, 29 too slowly, too quickly, excessively or insufficiently. This results then in lesions, which are still present during the second examination in block 38 and which can be determined by treatment monitor 25 and transferred into a correction of the transformation rule. The treatment device 10 can, therefore, be configured to determine and provide and use treating-person-specific transformation rules (transformation models), so that each treating person achieves a desired treatment result, independent from his/her individual skills. It is, however, also possible to provide treatment models based on the work of highly specialized and very successful treating persons for use by less specialized treating persons. In this manner treatment models can be transferred from specialized hospitals to less specialized hospitals and there also lead to good treatment successes.

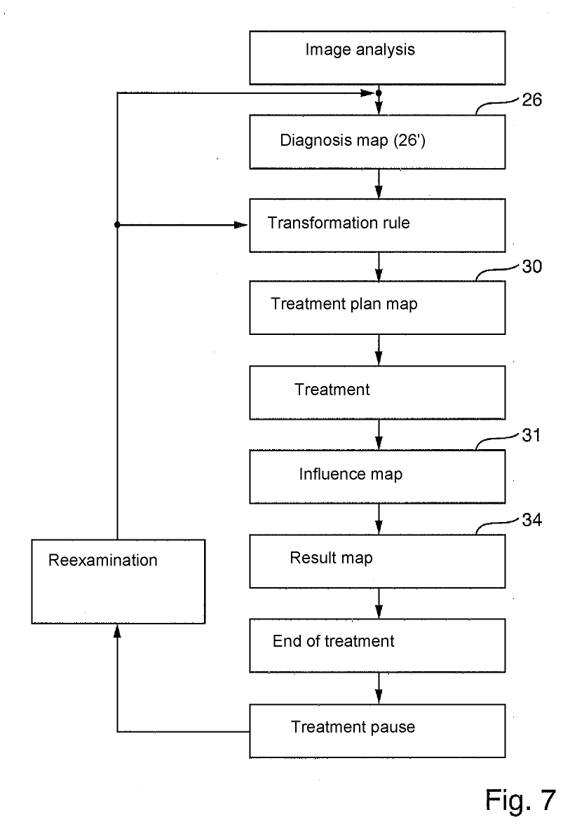

For further illustration of the optimization method for improving the diagnosis and treatment quality of cervical intraepithelial neoplasia according to the invention, reference is made to FIG. 7. At the beginning the examination of a patient by means of image analysis takes place. The diagnosis device 15 is configured to analyze captured images and to create a diagnosis map 26 therefrom. The diagnosis map 26 is transferred into the treatment plan map 30 by means of a transformation rule (treatment model). It follows the manual or automatic treatment of tissue 11 based on the treatment plan map 30 and subsequently under registration of the carried out treatment the creation of an influence map 31 that documents the treatment (influence), which has been carried out. Influence map 31 changes dynamically during the influence. Is the treatment finished, the influence map 31 contains the locally different influences. Only in the ideal case they are identical with the demand of the treatment plan map 30 and thus the result map 34 is empty.

With this the treatment ends in the first instance and typically a treatment pause of multiple days up to multiple weeks is carried out. During a new examination—again with image analysis by means of the diagnosis device 15—a new diagnosis map 26′ and if necessary also a correction of the transformation rule (treatment model) is determined after which the above-mentioned blocks are executed again. Due to the mentioned adaption of the transformation rule, typical treatment-person-specific deviations between the treatment plan map 30 and the influence map 31 are minimized on one hand and patient-specific different requirements to the treatment intensity are considered on the other hand. This simplifies the optimization of the therapy proposal in form of the treatment plan map 30.

The devices according to the invention in different facilities can be connected with one another via a network and can exchange adapted transformation rules for patient-specific treatment with one another. In addition, the larger data set of the network devices can be used for better adaption of the transformation rules by means of machine learning methods.

The treatment device according to the invention allows a treatment of tissue with a treatment medium 23 that does not directly show a tissue trace with manual as well as machine instrument guidance and under adjustment of the treatment plan map, due to preceding treatments. The treatment plan map represents the transformation rules for the treating person. The concept according to the invention allows the adaption to different patients or patient collectives as well as also the individualization of the creation of the treatment plan map with regard to the using treating persons.

### LIST OF REFERENCE SIGNS

10 treatment device
11 cervix tissue
12, 13 lamellae
14 image
15 diagnosis device
16 image capturing device
17 images, video
18 application device
19 instrument
20 apparatus for supply of instrument 19
21 image capturing device/camera
22 navigation device
22a recording device
22b display device
23 treatment medium
24 data memory
25 treatment monitor
26 diagnosis map
26′ diagnosis map for second or further examination
27-29 zones in the diagnosis map 26
30 treatment plan map
31 influence map
32, 33 zones of influence having different intensity
34 result map
40 blocks

1. A treatment device (10) for treatment of human or animal tissue comprising:
a diagnosis device (15) having an image capturing device (16) for capturing at least one image (17) of the tissue (11), wherein the diagnosis device (15) is configured for identification of zones (27, 28, 29) having need for treatment,
an application device (18) comprising:
an instrument (19) configured for application of a treatment medium (23) on the tissue (11) to be treated,
an apparatus (20) connected with the instrument (19) in order to supply the latter configured for supplying the instrument (19) with an operating medium and energy, and
a navigation device (22) configured to determine a location and a desired influence intensity of the treatment medium (23) on the tissue (11).

2. The treatment device according to claim 1, wherein the diagnosis device (15) is configured to identify zones (27, 28, 29) of different lesions in the at least one image (17) captured by the image capturing device (16) and to generate a diagnosis map (26) in this manner.

3. The treatment device according to claim 2, wherein the navigation device (22) and/or the diagnosis device (15) is configured to assign an influence intensity to each point of the tissue (11) captured by the at least one image (17) and to create a treatment plan map (30) in this manner.

4. The treatment device according to claim 3, wherein the navigation device (22) and/or the diagnosis device (15) is configured to use a transformation rule for creation of the treatment plan map (30) from the diagnosis map (26).

5. The treatment device according to claim 4, wherein the navigation device (22) and/or the diagnosis device (15) is configured to detect temporal variations of colorization in the at least one image (17) created during a staining test and to assign a necessary influence intensity to each point of the tissue (11) captured by the images (17).

6. The treatment device according to claim 1, wherein the treatment medium (23) is a non-thermal plasma.

7. The treatment device according to claim 3, wherein the navigation device (22) comprises a display device (22b) that is configured to optically visibly represent the treatment plan map (30) in relation to the tissue (11) to be treated.

8. The treatment device according to claim 7, wherein the navigation device (22) comprises a monitoring device (21) configured for visual inspection of the tissue to be treated by a user and a display device (22b) configured to superimpose the treatment plan map (30) with the tissue (11) in an optically visible manner for the user.

9. The treatment device according to claim 1, wherein the navigation device (22) comprises a recording device (22a) by means of which a treatment dosage and/or an influence location resulting from the influence of the treatment medium (23) on the tissue (11) can be detected.

10. The treatment device according to claim 7, wherein the display device (22b) is configured to represent a treatment dosage in an influence map (31).

11. The treatment device according to claim 2, wherein the diagnosis device (15) is connected with a data storage (24) in order to store the diagnosis map (26) in a patient-specific manner.

12. The treatment device according to claim 11, wherein the diagnosis device (15) and/or the navigation device (22) is configured to create an influence map (31) and is connected with the data storage (24) in order to store the influence map (31) in a patient-specific manner.

13. The treatment device according to claim 11, wherein the diagnosis device (15) and/or the navigation device (22) is configured to create an influence map (31) and is connected with the data storage (24) in order to store the influence map (31) in a treating-person-specific manner.

14. The treatment device according to claim 12, wherein the data storage (24) is connected with a treatment monitor (25) that is configured to determine a treatment success based on a treatment plan map (30) determined at a first examination date, the influence map (31) and a treatment plan map (30) determined at a second examination date.

15. A method for creation of a treatment plan map (30) for treatment of human or animal tissue during which:
carrying out a first two-dimensional examination of the tissue (11) in a first examination step;
creating and storing a first treatment plan map (30) based on the first two-dimensional examination using a creation rule;
carrying out a first treatment according to the first treatment plan map (30) in a first treatment step;
carrying out a second two-dimensional examination of the tissue in a second examination step
creating and storing a second treatment plan map (30) based on the second two-dimensional examination of using the creation rule; and
determining a correction recommendation for the creation rule from the first and second treatment plan maps (30).
